(19) United States
(12) Patent Application Publication
(10) Pub. No.: US 2022/0110855 A1
(43) Pub. Date: Apr. 14, 2022

(54) TOPICAL BARRIER COMPOSITION FOR DERMAL APPLICATION

(71) Applicants: Doris Day, New York, NY (US); John E. Kulesza, Wethersfield, CT (US)

(72) Inventors: Doris Day, New York, NY (US); John E. Kulesza, Wethersfield, CT (US)

(21) Appl. No.: 17/498,881

(22) Filed: Oct. 12, 2021

Related U.S. Application Data
(60) Provisional application No. 63/090,334, filed on Oct. 12, 2020.

(51) Int. Cl. A61K 8/81; A61K 8/44; A61K 8/67; A61Q 1/00

(52) U.S. Cl. CPC A61K 8/8158; A61Q 1/00; A61K 8/67; A61K 8/44

(57) ABSTRACT

Provided are dermal barrier compositions that create a barrier between external irritants or coverings and the skin of a subject upon which the composition is applied. A dermal barrier composition as provided herein includes hydrophilic polymer or copolymer and a fatty acid-amino acid conjugate. Also provided are methods of producing a dermal barrier composition. Also provided are methods of using a dermal barrier composition to maintain makeup on the skin of a subject.

### CROSS REFERENCE TO RELATED APPLICATION

This application depends from and claims priority to U.S. Provisional Patent Application No: 63/090,334 filed Oct. 12, 2020, the entire content of which is incorporated herein by reference.

### FIELD

The present disclosure concerns compositions that provide dermal barrier to shield skin from irritation and/or inflammation.

### BACKGROUND

The skin is exposed to many foreign substances, particles, particulates and objects on a daily basis, both by choice and by general exposure to the atmosphere. For example, humans voluntarily apply make-up, sunscreen, face creams and the like. The air itself can contain any number of chemical compounds and potential hazards and even wind can abrade the skin. While the skin serves to keep foreign irritants out of the body, it is susceptible itself to irritation. Occupations and health hazards can require a subject to place a face mask around the mouth and nose to filter the air and/or contain moisture from respiration. With increased use of face masks in the general public, more instances of perioral dermatitis or acne are seen as a result of the continued presence of the mask against the skin. For those that wear makeup such as lipsticks, blush, etc., wearing a mask can lead to rubbing off of the makeup leading to a visually compromised mask and reduced pleasing appearance of the wearer.

Other irritants can come from persons themselves as well as from outside sources. For example, when a person exhales the breath contains many components such as microorganisms and irritants that can be found in the saliva and nasal passages. These commonly land back on the skin of the subject's face and body. When wearing a mask these irritants are concentrated on the skin around the nose and mouth due to them being trapped by the mask. Even short periods of mask wearing can alter the microenvironment of the skin altering the skin microbiome and/or causing irritation that often in conjunction to physical rubbing of the mask on the skin may lead to skin breakdown allowing microorganisms to more readily penetrate the skin barrier.

In order to better protect the skin, a dermal barrier composition can serve to shield skin and prevent these conditions and issues from arising. As such, there is a need for new compositions to serve as a dermal barrier.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Provided are dermal barrier compositions that have the ability to protect the skin of a subject or other materials on the skin (e.g. makeup) of a subject from environmental abuses such as but not limited to those caused by the wearing of a mask or exposure to one or more environmental challenges. It was found that combining a hydrophilic polymer or copolymer with a fatty acid-amino acid conjugate produced a composition capable of resisting these physical or environmental stresses that the skin may be otherwise directly exposed to. As such, dermal barrier compositions are provided that include a hydrophilic polymer or copolymer and a fatty acid-amino acid conjugate. While the disclosure is not so limited, in some examples a hydrophilic copolymer is or includes poly(N,N-dimethylacrylamide-co-acrylic acid-co-polystyrene ethyl methacrylate) and a fatty acid-amino acid conjugate is or includes lauryl lysine. The composition optionally further includes one or more additional agents that may serve complementary functions to improve the look or feel of the composition or provide other physiological attributes to the system that may complement the combination of the hydrophilic polymer or copolymer and a fatty acid-amino acid conjugate.

Also provided are methods of forming a dermal barrier composition including: (a) preparing a first phase comprising a soothing agent, an antimicrobial agent, a hydro-enhancer, a sebum inhibitor, or a combination thereof with water and a preservative; (b) mixing the first phase with a second phase comprising a topical vitamin, a stabilizer, a surfactant, or a combination thereof; (c) mixing a third phase comprising a slip agent, an emulsifier, a barrier lipid, or combination thereof, and lauroyl lysine with the mixture from (b); and (d) adding a fourth phase of poly(N,N-dimethylacrylamide-co-acrylic acid-co-polystyrene ethyl methacrylate) to (c) and homogenizing to provide the dermal barrier composition.

Also provided are methods of protecting the skin of a subject and/or maintaining makeup on the skin of the subject that include applying the dermal barrier composition as provided herein to the skin of a subject, to a mask or other physical device that contacts the skin, or on the surface of makeup previously placed on the skin of the subject.

### DETAILED DESCRIPTION

The present disclosure concerns compositions that provide a dermal barrier between potential external irritants and or coverings and a subject's skin. Dermal barrier compositions include a hydrophilic polymer or copolymer and a fatty acid-amino acid conjugate. It was found that the specific combination of these materials, optionally along with other suitable agents that may serve complimentary functions in the dermal barrier compositions, can protect the skin against unwanted environmental, physiological, or mechanical stresses that otherwise may lead to irritation or undesirable outcomes. The dermal barrier compositions may be applied to the skin of a subject to protect the various layers of the dermis and may, in some instances, serve as an intermediate layer between underlying skin and/or makeup positioned on the outer epidermal layer, and thereby separate the skin from the environment or a covering. In some aspects, the dermal barrier composition forms a shield when topically applied. The dermal barrier composition, in some aspects, may include additional elements that can nourish and/or repair irritated or injured tissue. Optionally, the dermal barrier compositions can serve to prevent unwanted removal, smearing, or defacement of makeup on a subject's skin.

Dermal barrier compositions as provided herein include a hydrophilic polymer or copolymer. In some aspects, the hydrophilic copolymer may be a bioadhesive. As used herein, a bioadhesive may refer to a natural or synthetic material that can adhere to a biological surface, such as skin. In some aspects, a bioadhesive may form a continuous or discontinuous film on a biological surface. By way of example, a bioadhesive may include hydroxypropyl methylcellulose, ethyl cellulose, hydroxypropyl cellulose, polyvinyl pyrrolidine, polyvinyl alcohol, chitosan, polymethacrylate copolymers, silicones, polydimethylsiloxanes, acrylate copolymers, octylacrylamide copolymer, or octisalate. Examples of further copolymers that may be used as provided herein are set forth in U.S. Pat. No. 5,814,329 and US published patent application 2012/0157536.

In some aspects, the hydrophilic copolymer may include a water soluble polymer backbone chain with water insoluble styrenic polymer segments attached thereto. The main chain or backbone may include monomeric units having acidic groups and optionally neutral monomeric units. The acidic comonomers suitable for preparation of the copolymer may include acrylic acid, methacrylic acid, itaconic acid, 2-acrylamido-2-methyl-propane sulfonic acid, and 2-sulfoethyl methacrylate. Exemplary neutral comonomers may include acrylamide, methacrylamide, 2-hydroxyethyl methacrylate, N,N-dimethylacrylamide, polyethylene glycol monomethacrylate, and glyceryl methacrylate. In some aspects, the hydrophilic copolymer may include a copolymer of the formula:

[—(CH₂—CH-BLOCK A)ₓ—(CH₂—CH-BLOCK B)_{y}—(CH₂—C(BLOCK C)_{z}—CH₃],ₙ (Formula I)

wherein BLOCK A is O═C—N(CH₃)₂, BLOCK B is O═C—OH and BLOCK C is O═C—O—CH2-CH₂—[C(C₆H₅)H—CH₂]ₘ—R, wherein x, y, z, n, and m are positive integers of between 1 and 100 and R is an alkyl radical of 1 to 20 carbons. In some aspects, the hydrophilic copolymer may include poly(N,N-dimethylacrylamide-co-acrylic acid-co-polystyrene ethyl methacrylate). In some aspects, the hydrophilic copolymer is available as the compound PHARMADUR by Polytherapeutics, Inc. of New Jersey.

The hydrophilic polymer or copolymer may be present at from about greater than 0 to about 10 percent by weight of the dermal barrier composition, or any value or range therebetween. Optionally, the hydrophilic copolymer may be present at from about 0.1 to about 5 percent of the dermal barrier composition, optionally about 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. In some aspects, the dermal barrier composition may be present in the dermal barrier composition by weight percentage from about 0.1 to about 5 of poly(N,N-dimethylacrylamide-co-acrylic acid-co-polystyrene ethyl methacrylate).

The dermal barrier composition may include in addition to a hydrophilic copolymer, a fatty acid-amino acid conjugate in the form of a lipoamino acid or a lipopeptide. A peptide as provided herein is two or more amino acids linked by a peptide bond. The amino acids in a lipoamino acid or lipopeptide may be linked by peptide bonds to form a lipopeptide or may be isolated along the length or at an end of the fatty acid backbone, or combinations thereof. Amino acids can be used in either or both L and D stereoisomer forms. Amino acids may include proteogenic amino acids and non-proteogenic amino acids. Proteogenic amino acids may include alanine, arginine, glutamine, leucine, isoleucine, tryptophan, tyrosine, serine, threonine, lysine, methionine, phenylalanine, histidine, glutamic acid, aspartic acid, asparagine, cysteine, glycine, proline, selenocysteine, pyrrolysine, or valine.

Fatty acids used to form the conjugate may include substituted, unsubstituted, saturated and/or unsaturated carbon chains of between 2 to 40 carbon atoms in length, optionally 10 to 20 carbon atoms in length, optionally 12 to 18 carbon atoms in length. Saturated fatty acids may include propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, noncosylic acid, melissic acid, hentriacontylic acid, lacceroic acid, psyllic acid, geddic acid, ceroplastic acid, hexatriacontylic acid, heptatriacontylic acid, octatriacontylic acid, nonatricontylic acid and tetracontylic acid.

Unsaturated derivatives of a fatty acid may also be used to form a conjugate, such as with 1, 2, 3, 4, 5, 6, 7, 8, 9, 10, 11, or 12 double bonds between carbons. Examples of unsaturated fatty acids include octenoic, decenoic, lauroleic, myristovaccenic, myristolinoleic, myristolinolenic, palmitolinolenic, palmitidonic, alpha-linolenic, stearidonic, dihomo-alpha-linolenic, eicosatetraenoic, eicosapentaenoic, clupanodonic, docosaheaenoic, tetracosapentaenoic, tetracosahexaenoic, myristleic, palmitovaccenic, alpha-eleostearic, beta-eleostearic, punicic, octadecatrienoic, eicosatrienoic, eicosatetraenoic, tetradecenoic, octadecenoic, linoleic, linolelaidic, gamma-linolenic, calendic, pinolenic, dihomo-linolenic, dihomo-gamma-linoleic, arachidonic, adrenic, palmitoleic, vaccenic, rumenic, paulinic, oleic, eladic, gondoic, erucic, nervonic, sapienic, gadoleic and petroselinic acids.

In some aspects, the fatty acid-amino acid conjugate may include a lipoamino acid or a lipopeptide of lauric acid and lysine or poly-lysine. In some aspects, the fatty acid-amino acid conjugate may include lauroyl lysine.

In some aspects, a fatty acid-amino acid conjugate may be present in the composition at from about greater than 0 to about 10 percent by weight of the dermal barrier composition. In other aspects, the fatty acid-amino acid conjugate may be from about 0.1 to about 5 percent of the dermal barrier composition, including about 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. In some aspects, the dermal barrier composition may include by weight percentage from about 0.1 to about 5 of lauroyl lysine.

As set forth herein, an optional mixture of a hydrophilic backbone and hydrophobic segments along the length of a hydrophilic polymer or copolymer thereof may provide a suitable environment to better solubilize or suspend a lipoamino acid or a lipopeptide. In some aspects, the dermal barrier composition may include poly(N,N-dimethylacrylamide-co-acrylic acid-co-polystyrene ethyl methacrylate) and a fatty acid-amino acid conjugate. In certain aspects, the dermal barrier composition may include poly(N,N-dimethylacrylamide-co-acrylic acid-co-polystyrene e thyl methacrylate) and lauroyl lysine. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate).

In further aspects, it can be of benefit to include in the dermal barrier composition one or more further agents to benefit the underlying skin, such that while the dermal composition shields the skin, it can further provide topical application of one or more additional components to nourish or repair the underlying skin, including the dermis, epidermis and/or subcutaneous tissue. Additional components may include one or more of an antimicrobial peptide, a soothing agent, a sebum inhibitor, a hydro-enhancer, a topical vitamin, a stabilizer, a surfactant, an emulsifier, a barrier lipid, a slip agent, and combinations thereof

The dermal barrier composition may include an anti-microbial peptide. Anti-microbial peptides may be of benefit to control microbe populations of the skin. For example, acne can be exacerbated by the presence of microbes on the surface of the skin and the dermal barrier composition can both shield the skin from further microbe attachment, as well as control any microbes already present on the skin. In some aspects, the anti-microbial peptide may comprise an oligopeptide of phenylalanine, lysine, leucine and alanine. In some aspects, the oligopeptide may be of from about 5 to about 20 amino acids in length, including 6, 7, 8, 9, 10, 11, 12, 13, 14, 15, 16, 17, 18, and 19 amino acids. In other aspects, the oligopeptide may be of about 15 amino acids in length. In some aspects, the oligopeptide may feature about 4 lysine residues, 5 alanine residues, 5 leucine residues and a phenylalanine residue. In certain aspects, the oligopeptide is Oligopeptide-10 with a CAS No. of 466691-40-7.

The anti-microbial peptide may be present in the dermal barrier composition in a weight percentage of from about 0 to about 20, including, about 0.0001, 0.001, 0.01, 0.1, 1, 2, 3, 4, 5, 6, 7, 8, 9, 10, 11, 12, 13, 14, 15, 16, 17, 18, and 19. In some aspects, the dermal barrier composition may include from about 0.0001 to about 10 percent by weight of an anti-microbial peptide. In some aspects, the dermal barrier composition may include from about 0.0001 to about 10 percent by weight of Oligopeptide-10. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 0.001 to about 10 percent by weight of an anti-microbial peptide.

In some aspects, an additional component may include a barrier lipid. Barrier lipids may provide to the skin extra lipids for incorporating into cell walls to strengthen the skin and control permeability into the cells of the skin. Barrier lipids may include cholesterols, fatty acids and ceramides. Fatty acids may include an unsaturated and saturated fatty acid as described herein. Ceramides may include any compound of a sphingosine and a fatty acid of between 14 and 26 carbons in length. Cholesterols may include cholesterol itself and derivatives containing a 3-hydroxylated cholestane core. In some aspects, a barrier lipid component of the dermal barrier composition may include at least one of a ceramide, a cholesterol or a fatty acid. In other aspects, the dermal barrier composition may include a barrier lipid component of either a fatty acid and a ceramide, a fatty acid and a cholesterol or a cholesterol and a ceramide. In other aspects, a barrier lipid component may include one or more ceramides, one or more cholesterols and one or more fatty acids.

The barrier lipid component may be present in the composition at from about 0 to about 20 percent by weight of the dermal barrier composition, including, about 1, 2, 3, 4, 5, 6, 7, 8, 9, 10, 11, 12, 13, 14, 15, 16, 17, 18, and 19 percent. In some aspects, the dermal barrier composition may include from about 1 to about 10 percent by weight of a barrier lipid component. In some aspects, the dermal barrier composition may include from about 1 to about 10 percent by weight of a blend of ceramide, cholesterol and fatty acids. The dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 1 to about 10 percent by weight of a barrier lipid component.

Optionally, the additional agent may include a soothing agent. A soothing agent may include aloe vera, calendula, chamomile, blue tansy oil, colloidal oats, evening primrose oil, niacinamide, sea buckthorn oil, and allantoin. In certain aspects, the soothing agent may include aloe vera. Optionally, a soothing agent is allantoin. Optionally, a composition includes both aloe vera and allantoin.

The soothing agent may be present in the dermal barrier composition at from about from about 0 to about 20 percent by weight, including, about 1, 2, 3, 4, 5, 6, 7, 8, 9, 10, 11, 12, 13, 14, 15, 16, 17, 18, and 19 percent. Optionally, the dermal barrier composition may include from about 1 to about 10 percent by weight of a soothing agent. In some aspects, the dermal barrier composition may include from about 1 to about 10 percent by weight of aloe vera. In some aspects, the dermal barrier composition may include from about 0.1 to about 1 percent by weight of allantoin. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 1 to about 10 percent by weight of a soothing agent.

In some aspects, the dermal barrier composition may include an additional component of a sebum inhibitor. Sebum excretion can be associated with varying skin disorders and disruptions, including acne. In some instances, sebum inhibitors can include olumacostat glasretil, fullernol, glucocorticosteroid(s), and pyrrolidione carboxylic acid (PCA) and salts thereof, including a zinc PCA. In certain aspects, the sebum inhibitor is PCA or a salt thereof. In some instances, the salt may include a zinc salt.

A sebum inhibitor may be present at from about 0 to about 5 percent by weight of the dermal barrier composition, including about 0.1, 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. In some aspects, the sebum inhibitor is of from about 0.1 to 1 percent by weight of the dermal barrier composition. In some aspects, the dermal barrier composition may include from about 0.1 to about 1 percent by weight of PCA or zinc PCA. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 0.1 to about 1 percent by weight of a sebum inhibitor.

In some aspects, the additional component of the dermal barrier composition may include a hydro-enhancer. A hydro-enhancer may improve the water retention of a dermal cell. In some aspects, a hydro-enhancer may include hyaluronic acid. A hydro-enhancer may be present at from about 0 to about 5 percent by weight of the dermal barrier composition, including about 0.1, 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. In some aspects, the hydro-enhancer is of from about 0.1 to 1 percent by weight of the dermal barrier composition. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 0.1 to about 1 percent by weight of a hydro-enhancer.

Optionally, the dermal barrier composition may include an additional component of a topical vitamin. Topical vitamins may include retinoids, vitamin c, vitamin E, pantothenic acid and/panthenol, vitamin D, and vitamin K1. In some aspects, the topical vitamin is panthenol. A topical vitamin may be present in the dermal barrier composition at from about from about 0 to about 20 percent by weight, including, about 1, 2, 3, 4, 5, 6, 7, 8, 9, 10, 11, 12, 13, 14, 15, 16, 17, 18, and 19 percent. In some aspects, the dermal barrier composition may include from about 1 to about 10 percent by weight of a topical vitamin. In some aspects, the dermal barrier composition may include from about 1 to about 10 percent by weight of panthenol. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 1 to about 10 percent by weight of a topical vitamin.

In some aspects, the dermal barrier composition may include an additional component of a stabilizer. In some aspects, a stabilizer may include xanthan gum sodium gluconate, tetrasodium glutamate diacetate, butylated hydroxytoluene (BHT), ethylene diamine tetra acetic acid (EDTA), guar gum, gelatin, psyllium, or combinations thereof. In some aspects, the stabilizer is xanthan gum.

The stabilizer may be present at from about 0 to about 5 percent by weight of the dermal barrier composition, including about 0.1, 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. In some aspects, the stabilizer is of from about 0.1 to 1 percent by weight of the dermal barrier composition. In some aspects, the dermal barrier composition may include from about 0.1 to about 1 percent by weight of xanthan gum. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 0.1 to about 1 percent by weight of a stabilizer.

Optionally, the dermal barrier composition may include a slip agent. A slip agent may include talc, silicones or silicone derivatives, hexylene glycol, sorbitol, hydrolyzed silk, xylitol, zinc stearate, cetearyl olivate, or combinations thereof. Silicones and derivative are illustratively cyclic silicones or non-cyclic silicones. Examples of cyclic silicones illustratively include cyclic polydiorganosiloxanes, cyclotetradimethicones and cyclopentadimethicones. Linear organopolysiloxanes are illustratively alkyl-, alkoxy- or phenyldimethicones, and alkyl-, alkoxy- or phenyltrimethicones. Silicone derivatives may include cyclomethicones, dimethicone, trimethylsiloxysilicate, triethoxycaprylylsilane, simethicone, siloxane, silicone, silica dimethyl silylate, stearyl methicone, stearyl dimethicone, methyl trimethicone, decamethylcyclopentasiloxane, dodecamethylcyclohexasiloxane, and methicone. In some aspects, the slip agent is a cyclomethicone and/or dimethicone. In certain aspects, the slip agent is a combination of cyclomethicone and dimethicone.

The slip agent may be present at from about from about 0 to about 30 percent by weight of the dermal barrier composition, including, about 1, 2, 3, 4, 5, 6, 7, 8, 9, 10, 11, 12, 13, 14, 15, 16, 17, 18, 19, 20, 21, 22, 23, 24, 25, 26, 27, 28 and 29 percent. Optionally, the dermal barrier composition may include from about 1 to about 10 percent by weight of a dimethicone. In some aspects, the dermal barrier composition may include from about 1 to about 10 percent by weight of a cyclomethicone. In some aspects, the dermal barrier composition may include from about 1 to about 10 percent by weight of a dimethicone and from about 1 to about 10 percent by weight of a cyclomethicone. In some aspects, the dermal barrier composition may include from about 1 to about 10 percent by weight of a slip agent and/or from about 1 to about 20 percent of more than one slip agents. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate), from about 1 to about 20 percent by weight of a slip agent.

The dermal barrier composition may optionally include a surfactant. Illustrative examples of a surfactant include anionic, cationic, amphoteric and non-ionic surfactants. Optionally, a surfactant may include polysorbates (including polysorbate 80), sodium lauryl sulfate, sodium laureth sulfate, taurates, olefin sulfates, sulfosuccinates, isethionates, cetrimonium chloride, stearalkonium chloride, sodium lauriminodipropionate, disodium lauroamphodiacetate, cetyl alcohol, stearyl alcohol, and cocamidopropylamine oxide. It will also be appreciated that slip agents and emulsifiers as described herein may further be utilized for surfactant properties. In certain aspects, the surfactant is the polysorbate 80 or Tween 80.

A surfactant may be present at from about 0 to about 5 percent by weight of the dermal barrier composition, including about 0.1, 0.2, 0.3, 0.4, 0.5, 0.6, 0.7, 0.8., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, and 4.9 percent by weight of the dermal composition. Optionally, the surfactant is of from about 0.1 to 1 percent by weight of the dermal barrier composition. In some aspects, the dermal barrier composition may include from about 0.1 to about 1 percent by weight of polysorbate 80. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 0.1 to about 1 percent by weight of a surfactant.

Optionally, the dermal barrier composition may include an emulsifier. In some instances, the emulsifier may be a nonionic emulsifier. In certain aspects, the emulsifier is selected for suitability for oil-in-water compositions. Emulsifiers may include cetyl, stearyl alcohol, eicosanol (C20), behenyl alcohol (C22), glyceryl stearate, steareth 2, sorbitan stearate, PEG40 stearate, steareth 20, cetearyl glucoside, polyglyceryl 2 stearate, polyglyceryl 3 stearate, sodium stearoyl glutamate, sodium lauroyl lactylate, distearyldimethyl ammonium chloride, polyglyceryl 10 oleate, polyglyceryl 10 stearate, C12-C13 pareth 9, sorbitan oleate decylglucoside crosspolymer, polyglyceryl 10 oleate, polyglyceryl 2 oleate, sucrose laurate, and glycerin. By way of example, an emulsifier may include a silicone based emulsifier. Optionally, a silicone based emulsifier may be sourced as an example as ABIL Care 85 by EVONIK. In some aspects, the emulsifier may include bis-PEG/PPG-16/16PEG/PPG-16/16 dimethicone and caprylic/capric triglyceride, cetyl PEG/PPG-10/1 dimethicone, cyclopentasiloxane and PEG/PPG-19/19-dimethicone, PEG/PPG-19 dimethicone and C13-16 isoparrafin and C10-13 isoparrafin, dimethicone. And PEG/PPG-18/18 dimethicone, cyclopentasiloxane and PEG/PPG-18/18 dimethicone, dimethicone and PEG/PPG-18/18 dimethicone, lauryl PEG/PPG-18/18 methicone, cyclopentasiloxane and PEG-12 dimethicone crosspolymer, bis-isobutyl PEG/PPG-10/dimethicone crosspolymer, lauryl PEG-10 tris(trimethylsiloxysilyethyl dimethicone crosspolymer, PEG-10 dimethicone, cetyl diglyceryl tris(trimethylsiloxysilyethyl dimethicone, PEG-8 dimethicone, PEG-10 dimethicone, lauryl PEG/PPG-18/18 dimethicone, PEG-12 dimethicone, PEG-8 dimethicone, bis PEG/PPG-14/14 dimethicone and dimethicone, silicone quaternium-22 and polyglyceryl-3 and caprate and dipropylene glycol and cocamidopropyl betaine, quternium-80 and dipropylene glycol, methoxy PEG/PPG-7/3 aminopropyl dimethicone, polysilicone 19, and cetyl dimethicone. In some aspects, the emulsifier is bis-PEG/PPG-16/16PEG/PPG-16/16 dimethicone and caprylic/capric triglyceride.

The dermal barrier composition may include an emulsifier from about from about 0 to about 20 percent by weight, including, about 1, 2, 3, 4, 5, 6, 7, 8, 9, 10, 11, 12, 13, 14, 15, 16, 17, 18, and 19 percent. In some aspects, the dermal barrier composition may include from about 1 to about 10 percent by weight of an emulsifier. Optionally, the dermal barrier composition may include from about 1 to about 10 percent by weight of bis-PEG/PPG-16/16PEG/PPG-16/16 dimethicone and caprylic/capric triglyceride. In some aspects, the dermal barrier composition may include by weight percentage from about 0.1 to about 5 of lauroyl lysine and from about 0.1 to about 5 of poly(N,N-dimethylacrylamide-co-acrylic acid-co-polystyrene ethyl methacrylate) and from about 1 to about 10 percent by weight of an emulsifier.

In further aspects, the dermal barrier composition may include one or more preservatives. Such preservatives may be included in small amounts within the dermal composition, such that they may be added or included for the purposes of the amount need or quantum satis in order to provide the desired texture, color and/or consistency. Preservatives, by way of example, may include triclosan, triclocarban, phenoxyethanol, benzyl benzoate, methylisothiazolinone, zinc pyrithione, benzalkonium chloride, benzyl alcohol, and essential oils.

In further aspects, the dermal barrier composition may include water. Water may be added as needed during the preparation of the composition as the user sees needed for a particular consistency, texture and/or feel.

In further aspects, the dermal barrier composition may include from about 0.1 to about 5 weight percent of lauroyl lysine and from about 0.1 to about 5 weight percent of poly(N,N-dimethylacrylamide-co-acrylic acid-co-polystyrene ethyl methacrylate) and/or from about 1 to about 10 percent by weight of an emulsifier and/or from about 0.1 to about 1 percent by weight of surfactant and/or from about 1 to about 20 percent by weight of one or more slip agents and/or from about 0.1 to about 1 percent by weight of a stabilizer and/or from about 1 to about 10 percent by weight of a topical vitamin and/or from about 0.1 to about 1 percent by weight of a hydro-enhancer and/or from about 0.1 to about 1 percent by weight of a sebum inhibitor and/or from about 1 to about 10 percent by weight of an anti-microbial peptide and/or from about 1 to about 10 percent by weight of a barrier lipid component of a blend of a barrier lipid and/or from about 1 to about 10 percent by weight of a soothing agent and/or water and/or a preservative.

Further included in the dermal barrier composition may be one or more other bioactive agents. In certain aspects, a user may add in a bioactive agent prior to topical application. In some aspects, bioactive agents may be those that provide benefit when applied topically. Illustrative examples may include: hydroxy acids; aromatic molecules such as benzoyl peroxide and resorcinol; antimicrobials such as azelaic acid, erythromycin, sodium sulfacetamide, tetracycline and derivatives, and clindamycin; anti-neoplastic agents and/or ophthalmic agents illustratively including 5-fluorouracil, doxorubicin, imiquimod, and sodium [o-(2,6-dichloranilino) phenyl] acetate; anti-viral agents illustratively ganciclovir, trifluorothymidine and related compounds; steroidal anti-inflammatory agents; nonsteroidal anti-inflammatory agents illustratively flurbiprofen, ibuprofen, naproxen, indomethacin and related compounds; anti-mitotic drugs illustratively colchicine taxol and related compounds; drugs that act on actin polymerization illustratively phalloidin, cytochlasin B and related compounds; inhibitors of dihydropyrimidine dehydrogenase (DPD), thymidine phosphorylase (TP) and/or uridine phosphorylase (UP) enzyme inhibitors; ultraviolet light (UV) filters illustratively benzophenone derivatives such as oxybenzone, octocrylene, octyl methoxycinnamate, and avobenzone; radiation proactive agents illustratively methyluracils such as 6-methyluracil and 4-methyluracil; and immunomodulating molecules such as tacrolimus, and pimecrolimus; peptides (i.e. non-enzymatic protein); enzymes; amino acids, illustratively glutamate, glycine, alanine, valine, leucine, isoleucine, serine, threonine, arginine, lysine, aspartic acid, methionine, phenylalanine, tyrosine, proline, oxyproline, hidtidine, among other amino acids, both essential an non-essential known in the art, and derivatives thereof; growth factors, illustratively, epidermal growth factor, TGF-α, TGF-β, VEGF, among others known in the art; topical anesthetics, illustratively, benzocaine, butamben, dibucaine, lidocaine, oxybuprocaine, pramoxine, proparacaine, proxymetacaine, and tetracaine; niacinamide; functional metal elements such as silver that may be part of a composition such as silver sulfadiazine, and combinations thereof. A bioactive agent need not have pharmaceutical activity. Other bioactive agents are illustratively cosmetics such as pigments, dyes, and fillers.

A dermal barrier composition may be in the form of a liquid or solid solution, an oil in water emulsion, a water in oil emulsion, or other suitable form. A dermal barrier composition may be applied to a surface, illustratively the surface of a mask that is intended for wearing or other contact with the skin, or applied directly to the skin of a user. A mask may be a cloth or other material face shield that is worn over the mouth and/or nose of a subject. Optionally, a mask is a medical mask such as those used for a ventilator of continuous positive airway pressure (CPAP) therapy device. Optionally, any device or system that comes into contact with the skin of a subject may have a dermal barrier composition applied thereto or the dermal barrier composition may be applied to the skin that is or will be in contact with the device or system.

The dermal barrier composition may in some aspects be applied to the skin of a subject. A subject is optionally a patient. A subject is optionally a mammal such as humans, non-human primates, horses, goats, cows, sheep, pigs, dogs, cats, and rodents.

The dermal barrier composition may in some aspects be provided as a lotion, cream, gel, bar, ointment, spray, roller ball applicator, or in pad form. Optionally, the composition is provided in a single use container the contents of which are applied directly to the stratum corneum of a subject, applied to an applicator pad for subsequent delivery to the subject, or in some aspects may be applied to a mask prior to use by a wearer. Application to a mask, optionally as a spray or other suitable application form, would help assist in preventing accidental overspray to undesired areas (e.g. eyes), but also limits contact to areas directly covered by the mask for improved effectiveness. As such, administration may be by rubbing, spraying, or otherwise contacting the dermal barrier composition to the skin of a subject, or to a device or material that is then placed in contact with the skin.

The dermal barrier composition may in some aspects be administered one to three times daily. Optionally, the inventive composition is administered once daily. Optionally, the inventive composition is administered weekly, biweekly, monthly, or any subdivision thereof. It is appreciated that the inventive composition is optionally administered for an amount of time suitable for efficacy of the active agent. Optionally, the inventive composition is administered for one to six weeks. Optionally, the inventive composition is administered indefinitely.

Numerous skin or systemic conditions may be treatable, diagnosable, or preventable with the inventive compositions illustratively including acne, wrinkles, musculoskeletal pain, inflammation, dryness, eczema, psoriasis, actinic and nonactinic keratoses, rosaceous, among others, or combinations thereof.

In some aspects, a dermal barrier composition is used as a makeup protectant. In such instances, a dermal barrier composition may be applied to the skin on top of makeup so as to serve as a shield against environmental or physical abuse to the underlying makeup layer, optionally as is produced by wearing a mask on top of makeup. Any makeup that is typically applied to the face such as the oral or nasal regions may be protected such as but not limited to blush, foundations, lipstick, etc.

In further aspects, the present disclosure also includes methods of preparing the dermal barrier composition. Due to the hydrophilic and hydrophobic nature of the components, the methods may include one or more phases of combining and/or mixing. Optionally, a first component may include adding optional water and/or the soothing agent and/or the anti-microbial peptide and/or the hydro-enhancer and/or preservatives in a first phase. A second phase may include the topical vitamin, the stabilizer and the surfactant. A third phase may include the slip agent, the emulsifier, barrier lipids and the lipopeptide or lipoamino acid. The fourth phase may include the hydrophilic copolymer. Optionally, the first phase is mixed and all components are dissolved. The components of the second phase are then dispersed and mixed in to the first phase. Similarly, the third phase is then dispersed and mixed in to the combination of phase 1 and phase 2. The fourth phase may then be added and all homogenized together.

The presence of some emulsifiers will allow the process to occur at room temperature due to their texture and malleability. In other instances where a solid emulsifier may be used, heating of up to around 70° C. may be needed to mix the first three phases together. Addition of the hydrophilic copolymer in such cases would be performed after allowing to cool.

The compositions and methods described herein are presently representative of exemplary aspects, and not intended as limitations on the scope of the invention. Changes therein and other uses will occur to those skilled in the art. Such changes and other uses can be made without departing from the scope of the invention as set forth in the claims.

The foregoing description of particular aspect(s) is merely exemplary in nature and is in no way intended to limit the scope of the invention, its application, or uses, which may, of course, vary. The disclosure is presented with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

We claim:

1. A dermal barrier composition comprising a hydrophilic polymer or copolymer and a fatty acid-amino acid conjugate.

2. The dermal barrier composition of claim 1, comprising said hydrophilic copolymer.

3. The dermal barrier composition of claim 2 wherein said hydrophilic copolymer comprises or consists of formula I:
[—(CH₂—CH-BLOCK A)ₓ—(CH₂—CH-BLOCK B)_{y}—(CH₂—C(BLOCK C)_{z}—CH₃]ₙ (Formula I)
wherein BLOCK A is O═C—N(CH₃)₂, BLOCK B is O═C—OH and BLOCK C is O═C—O—CH₂—CH₂—[C(C₆H₅)H—CH₂]ₘ—R, wherein x, y, z, n, and m are positive integers of between 1 and 100 and R is an alkyl radical.

4. The dermal barrier composition of claim 2, wherein the hydrophilic copolymer comprises poly(N,N-dimethylacrylamide-co-acrylic acid-co-polystyrene ethyl methacrylate).

5. The dermal barrier composition of claim 1, wherein said fatty acid-amino acid conjugate comprises a fatty acid selected from the group consisting of propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, carboceric acid, montanic acid, noncosylic acid, melissic acid, hentriacontylic acid, lacceroic acid, psyllic acid, geddic acid, ceroplastic acid, hexatriacontylic acid, heptatriacontylic acid, octatriacontylic acid, nonatricontylic acid and tetracontylic acid.

6. The dermal barrier composition of claim 1, wherein said fatty acid-amino acid conjugate comprises a fatty acid selected from the group consisting of octenoic, decenoic, lauroleic, myristovaccenic, myristolinoleic, myristolinolenic, palmitolinolenic, palmitidonic, alpha-linolenic, stearidonic, dihomo-alpha-linolenic, eicosatetraenoic, eicosapentaenoic, clupanodonic, docosaheaenoic, tetracosapentaenoic, tetracosahexaenoic, myristleic, palmitovaccenic, alpha-eleostearic, beta-eleostearic, punicic, octadecatrienoic, eicosatrienoic, eicosatetraenoic, tetradecenoic, octadecenoic, linoleic, linolelaidic, gamma-linolenic, calendic, pinolenic, dihomo-linolenic, dihomo-gamma-linoleic, arachidonic, adrenic, palmitoleic, vaccenic, rumenic, paulinic, oleic, eladic, gondoic, erucic, nervonic, sapienic, gadoleic and petroselinic acid.

7. The dermal barrier composition of claim 1, wherein the fatty acid-amino acid conjugate comprises a fatty acid with one more lysine residues; or
wherein said fatty acid-amino acid conjugate comprises lauroyl lysine.

8. The dermal barrier composition of claim 1, wherein the hydrophilic copolymer is poly(N,N-dimethylacrylamide-co-acrylic acid-co-polystyrene ethyl methacrylate) present at from 0.1 percent to 5 percent by weight of the composition.

9. The dermal barrier composition of claim 1, wherein the hydrophilic copolymer is poly(N,N-dimethylacrylamide-co-acrylic acid-co-polystyrene ethyl methacrylate) and the fatty acid-amino acid conjugate is lauroyl lysine present from 0.1 percent to 5 percent by weight of the composition.

10. The dermal barrier composition of claim 1, further comprising an additional agent selected from the group consisting of a surfactant, an emulsifier, a topical vitamin, a hydro-enhancer, an anti-microbial peptide, a barrier lipid, a sebum inhibitor, a slip agent, a stabilizer, a soothing agent or a combination thereof

11. The dermal barrier composition of claim 10, wherein the soothing agent, the antimicrobial peptide, the topical vitamin, the emulsifier, the barrier lipid, or all of the foregoing is present at from 1 percent to 10 percent by weight of the composition.

12. The dermal barrier composition of claim 10, wherein the slip agent is present from 1 percent to 20 percent by weight of the composition.

13. The dermal barrier composition of claim 10, wherein the hydro-enhancer, the stabilizer, the sebum inhibitor, or all of the foregoing is present from 0.1 percent to 1 percent by weight of the composition.

14. A method of preparing a dermal barrier composition comprising:
(a) preparing a first phase comprising a soothing agent, an antimicrobial agent, a hydro-enhancer, a sebum inhibitor, or a combination thereof with water and a preservative;
(b) mixing the first phase with a second phase comprising a topical vitamin, a stabilizer, a surfactant, or a combination thereof;
(c) mixing a third phase comprising a slip agent, an emulsifier, a barrier lipid, or combination thereof, and lauroyl lysine with the mixture from (b); and
(d) adding a fourth phase of poly(N,N-dimethylacrylamide-co-acrylic acid-co-polystyrene ethyl methacrylate) to (c) and homogenizing to provide the dermal barrier composition.

15. A method of maintaining makeup on the skin of a subject comprising applying the composition of claim 1 to the skin of a subject.

16. The method of claim 15, wherein the applying is in the oral and or nasal region of the subject.

17. The method of claim 15, wherein the hydrophilic copolymer comprises poly(N,N-dimethylacrylamide-co-acrylic acid-co-polystyrene ethyl methacrylate).

18. The method of claim 15, wherein the fatty acid-amino acid conjugate comprises a fatty acid with one more lysine residues; or
wherein said fatty acid-amino acid conjugate comprises lauroyl lysine.

19. The method of claim 15, wherein the hydrophilic copolymer is poly(N,N-dimethylacrylamide-co-acrylic acid-co-polystyrene ethyl methacrylate) present at from 0.1 percent to 5 percent by weight of the composition.

20. The method of claim 15, wherein the hydrophilic copolymer is poly(N,N-dimethylacrylamide-co-acrylic acid-co-polystyrene ethyl methacrylate) and the fatty acid-amino acid conjugate is lauroyl lysine present from 0.1 percent to 5 percent by weight of the composition.
